(19) United States
(12) Patent Application Publication
(10) Pub. No.: US 2011/0184006 A1
(43) Pub. Date: Jul. 28, 2011

(54) METHOD AND COMPOSITION FOR THERMALLY STABILIZING VITAMIN C WITHIN NANO LAYERS OF MONTMORILLONITE CLAY

(75) Inventors: Chandhi S. Goonesekara, Kandy (LK); Vajira Seneviratne, Yakvila (LK); D. Nedra Karunaratne, Kandy (LK); Veranja Karunaratne, Kandy (LK)

(73) Assignee: NATIONAL RESEARCH COUNCIL OF SRI LANKA, Colombo (LK)

(21) Appl. No.: 12/696,048

(22) Filed: Jan. 28, 2010

(51) Int. Cl. A61K 31/375; A61K 31/192; A61K 31/19; A61K 31/194; A61K 31/198; A61K 31/513; A61P 3/02

(52) U.S. Cl. 514/274; 514/570; 514/557; 514/574; 514/562; 514/561; 514/474

(57) ABSTRACT

A thermally stable Vitamin C composition containing montmorillonite, an organic acid containing L-ascorbic acid or a derivative thereof, a divalent cation present within nanolayers of the montmorillonite, wherein the organic acid is combined with the divalent cation within the nanolayers to form an organic acid-montmorillonite chelate.

### BACKGROUND

Vitamins are an essential component of the human and animal diet in the metabolic processes of the body. Vitamin C is one such vitamin and is the general name for the L-ascorbic acid (LAA) and functions of Vitamin C are numerous. The most prominent role is its immune-stimulating effect, of great importance in defense against infections such as common colds. It also acts as an inhibitor of histamine, a compound that is released during allergic reactions, and as antioxidant to neutralize harmful free radicals to neutralize pollutants and toxins. LAA acts as a scavenger for oxidants and reactive oxygen species that are damaging to humans and plants at the molecular level by reducing them to water and biologically inactive species. During this process LAA is oxidized to L-dehydroascorbic acid that is relatively stable and unreactive. See FIG. 1 for reactions and structure of LAA. Pellets are the most common form of Vitamin C supplements available for ingestion. However, during pellet manufacture exposure to heat and light makes LAA unstable because it oxidizes to dihydroascorbate radicals. Another disadvantage is that LAA when present in aqueous alkaline solutions loses its color and stability.

Great Britain Patent 763098 disclosures a mixture of vitamins stabilized in compositions containing montmorillonite. Stable topical compositions containing ascorbic acid and a liquid emulsion phase with an organo clay material is disclosed in U.S. Pat. No. 5,902,591. U.S. Patent Application 20060078578 discloses stabilized film cosmetic compositions containing a stabilizer dispersed in non-quaternary montmorillonite. U.S. Patent Application 2008031960, discloses stabilized Vitamin C compositions containing caffeic acid.

Japanese Patent 5269184 (A) discloses porous structures formed by intercalation of an organic acid metal salt between the layers montmorillonite and saponite. Odorous materials having poor chemical reactivity were deodorized by exposing these materials to the catalytic effect of the organic acid metal salts by attracting them into the intercalated structure. Release of materials from these porous structures is not disclosed. Japanese Patent 9184836 (A) discloses detection of coloring material by stabilization with montmorillonite containing hydrogen peroxide and ascorbic acid.

Phosphoric acid treated montmorillonite to stabilize LAA is disclosed by Chen et al. See Yuan-Haun Lee, Bor-Yann Chen, Kun-Yu Lin, King-Fu Lin and Feng-Huei Lin, Journal of the Chinese Institute of Chemical Engineers, Volume 39, Issue 3, May 2008, Pages 219-226. Further studies of Lee et al., on montmorillonite-LAA nanocomposites concluded that L-ascorbate anions, LAA particles could be absorbed or intercalated within the montmorillonite layers, particularly in wider basal d-spacing at pH 7 to 10. Their studies were done with purified montmorillonitrile and the significant increase in d-spacing prompted the use montmorillonite as a drug carrier. See. B. Y. Chen, Y. H. Lee, W. C. Lin, F. H. Lin, K. F. Lin, 2006, ‘Understanding the characteristics of L-ascorbic acid-montmorillonite nanocomposite: chemical structure and biotoxicity’, Biomedical Engineering Applications, Basis and Communications, vol. 18, no. 1, pp 30-36. However, thermal stability of calcium montmorillonite-LAA of the present invention is not disclosed.

An animal feed containing an encapsulated Vitamin C that is mixed with ground montmorillonite for ruminant feeds is disclosed in WO2008015023. For poultry feeds, Vitamin C supplementation is required to alleviate temperature induced stress symptoms. See Damerow G., 1994, The Chicken Health Handbook, Storey publishing, USA. These supplements are usually administered in pellet form, where ascorbic acid is mixed with fillers and heat-treated at about 100° C.

Given the above, it is desirable to have a LAA chelated-montmorillonite that is thermally stable in processing the Vitamin C supplements. Also desired is a sustained release composition that provides LAA acid in pH range of about 1 to about 5 typically found in the stomach of a human. As such stability in the range of pH 6 to 9 found in the human intestine is desired for these LAA chelated montmorillonite. Further, such a feed material can serve as an animal feed supplement in release of LAA in the chicken gut having a pH of about 1 to about 4. A process that is relatively simple and involves use of water soluble or dispersible non-toxic materials is industrially attractive in manufacturing Vitamin C supplements.

### SUMMARY

Accordingly disclosed herein is a thermally stable Vitamin C composition comprising montmorillonite, an organic acid comprising LAA or a derivative thereof, a divalent cation present within nanolayers of the montmorillonite. The organic acid is combined with the divalent cation present within the nanolayers of the monmorillonite and forms a thermally stable organic acid-montmorillonite chelate. Embodiment compositions contain LAA chelated to divalent calcium cations present within the nanolayers of montmorillonite. The structure of the chelate formed between calcium cation and L-ascorbate is shown hereinbelow:

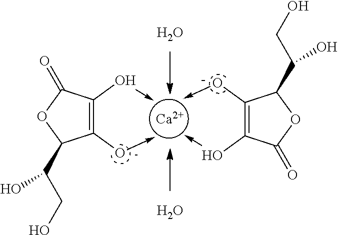

It is believed that deprotonation of LAA forms the enolate that is strongly basic and the resulting resonance structures are hereinbelow and in FIG. 1:

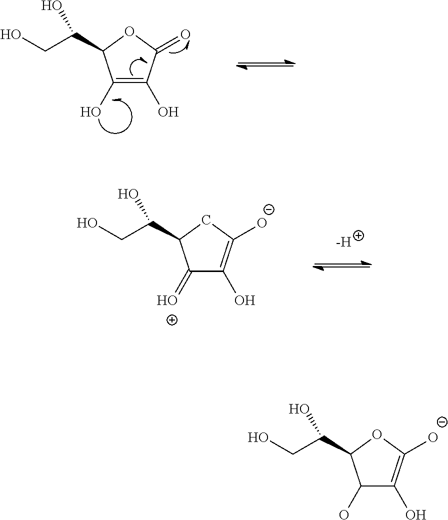

Any pharmaceutically active divalent, trivalent or multivalent cation that can form chelates with LAA within the nanolayers of montmorillonite can be used. Suitable organic acids that can form chelates within the nanolayers of the montmorillonite are chosen from a member selected from the group consisting of ascorbic acid, caffeic acid, quinic acid, chlorendic acid, citric acid, methionine, cysteine, malic acid, dimercapto succinic acid, aspartic acid, orotic acid, and mixtures thereof. Embodiment divalent cations are selected from a member of the group consisting of calcium, magnesium, zinc, and barium, and combinations thereof.

The LAA-montmorillonite chelate can be used as carrier for Vitamin C supplements. The release rates of LAA obtained at various pH ranges found in the human gastrointestinal tract indicates that this chelate can be used as a controlled-release or sustained release drug carrier. This chelate prevents is oxidative degradation and releases LAA in a sustained manner at pH values from about 1 to about 5. Such an released LAA retains its antioxidant stability. It has been surprisingly found the LAA-montmorillonite chelate was stable at higher pH values and had the maximum stability at pH of about 9. It is believed that at least 95% of the chelate is formed around pH of about 9 consistent with the stability of LAA enolate. Such stability makes this chelate suitable for oral ingestion as rapid oxidative degradation occurring in the human duodenum at pH of about 6 can be minimized. Also disclosed is a method of delivering therapeutic amounts of Vitamin C to a human that comprises orally administering the LAA-montmorillonite chelate. In an embodiment the organic acid-montmorillonite chelate is LAA-montmorillonite and the divalent cation is calcium. Another embodiment comprises sustained release of Vitamin C in the stomach of a human in the pH range from about 1 to about 5. These compositions can further comprise a filler and pharmaceutically acceptable excipients.

In an embodiment the organic acid-montmorillonite chelate is stabilized in the temperature range from about 90° C. to about 200° C. In another embodiment the organic acid-montmorillonite chelate is stabilized in the temperature range from about 140° C. to about 200° C.

In another embodiment the LAA-montmorillonite chelate can be used to prepare Vitamin C supplements to be administered in domestic fowl. Such supplements could be administered orally and enhanced thermal stability of the LAA-calcium montmorillonite chelate compared to LAA makes the chelate suitable manufacture of such fowl feed supplements.

Also disclosed is a process for preparing a thermally stable Vitamin C composition comprising:
a. contacting an organic acid comprising L-ascorbic acid or a derivative thereof with a montmorillonite containing a divalent cation present within nanolayers of the montmorillonite;
b. combining the organic acid with the divalent cation to form an organic acid-montmorillonite chelate; and
c. stabilizing the organic acid-montmorillonite chelate in the temperature range from about 90° C. to about 200° C.

### BRIEF DESCRIPTION OF THE DRAWINGS

FIG. 1: Structures and electronic configurations of L-ascorbic acid.

FIG. 2: Chemical structure of montmorillonite.

FIG. 3: X-ray diffraction spectra for calcium montmorillonite at various pH values.

FIG. 4: X-ray diffraction spectra for LAA-calcium montmorillonite chelates at pH values 2, 5, 7, 9 and 11.

FIG. 5: TGA/DSC thermograms for calcium montmorillonite.

FIG. 6: TGA/ DSC plots of calcium montmorillonite and LAA-calcium montmorillonite chelates (mass ratio LAA:calcium montmorillonite at 1:5) at pH values of 7, 9 and 11.

FIG. 7: FTIR spectra of calcium montmorillonite and LAA-calcium s montmorillonite chelates (LAA:montmorillonite mass ratio of 1:5) at pH values of 2, 5, 7, 9 and 11.

FIG. 8: FTIR spectra of (a) calcium montmorillonite and LAA-calcium montmorillonite chelate at pH of 9.0; and (b) calcium montmorillonite at pH of 9.0.

FIG. 9: Graph of the number of moles of LAA released from the chelate vs. time, at different pH values.

FIG. 10: FTIR spectra of LAA-calcium montmorillonite chelate and L-ascorbic acid, after heating at 100° C., 120° C., 140° C., 160° C., 180° C. and 200° C., for 30 minutes.

is FIG. 11: FTIR spectra of L-ascorbic acid before and after heating at 90° C. for 30 minutes.

FIG. 12: General structure of the digestive system of a human.

FIG. 13: General structure of the digestive system of a chicken.

FIG. 14: Graph of the postulated number of moles of LAA released from 0.25 g sample of LAA-calcium montmorillonite chelate along the human GI tract vs. time.

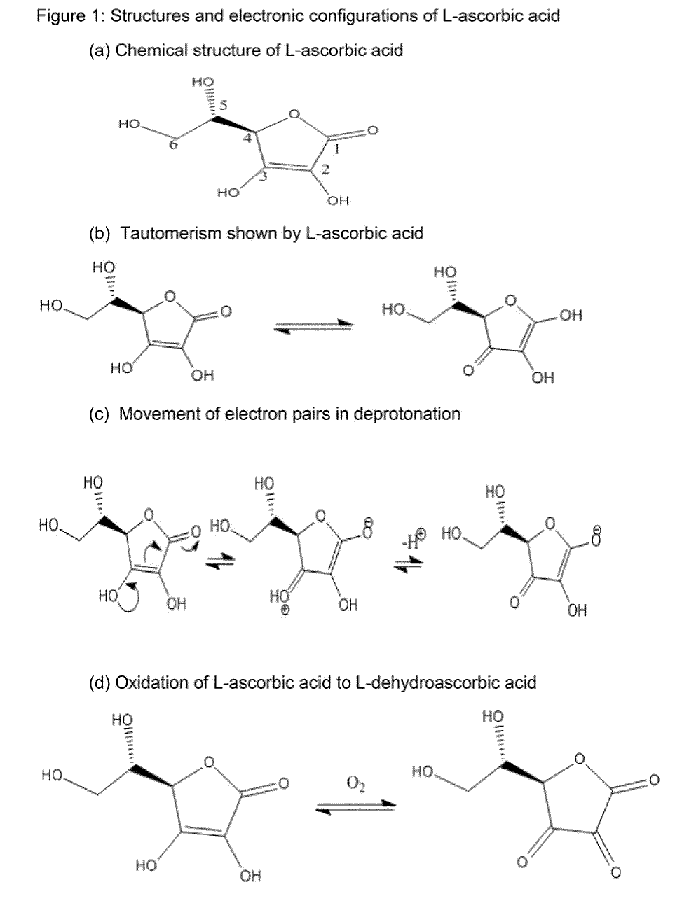

### DETAILED DESCRIPTION

### Definitions:

Vitamin C referred to herein is L-ascorbic acid and all the biologically active forms. Vitamin C comprises L-ascorbic acid and L-dehyroascorbic acid that are interconvertible via free radical intermediates known in the art.

Chelation as referred to herein is substantial covalent bond formation between the organic acid and the divalent cation. Substantial chelation as referred to herein is where at least 95% of the organic acid is chelated. In an embodiment at least 98% of the organic acid is chelated.

Divalent cations as referred to herein include multivalent cations. Examples of divalent cations include calcium, magnesium, barium, alumnium and cerium.

Organic acids as referred to herein include derivatives that are capable of chelating with the divalent cations. Suitable organic acids are selected from a member of the group consisting of ascorbic acid, caffeic acid, quinic acid, chlorendic acid, citric acid, methionine, cysteine, malic acid, dimercapto succinic acid, aspartic acid and orotic acid, and mixtures thereof. Derivatives of ascorbic acid are derivatives that are capable of chelating with the divalent cations.

Nanolayers as referred to herein are interlayers present between the crystal planes of montmorillonite. Water molecules and ion-exchangeable divalent cations are present within these interlayers. Nanolayer as referred to is herein includes the plural form.

Sustained release as referred to herein includes controlled release of LAA.

Chicken as referred to herein includes a ruminant.

Alkyl groups and alkoxy groups as referred to herein contain 1 to 20 linear or branched carbon atoms.

### Methods:

### (a) LAA Concentration

LAA concentrations were obtained using redox titration with iodine and absorbance measurements.

LAA concentrations of samples were obtained by titrating with a standard I₂/KI (aq) solution (0.005 mol dm⁻³) with starch indicator.

A standard aqueous stock solution having a concentration 0.05 mol dm⁻³ LAA was prepared and the pH was adjusted to pH of 1.0 with conc. HCl (aq). Dilutions were made with distilled water adjusted to pH of 1.0 to obtain solutions LAA with different concentrations. Absorbance of these solutions was measured using a UV-visible spectrophotometer at a wavelength of 245.0 nm. Using the above procedure, standard solutions of LAA were prepared at pH values of 3.0, 5.0, 7.0 and 9.0 and their absorbance values (λₘₐₓ) were obtained at wave lengths of 250.0 nm, 265.6 nm, 265.0 nm and 265.0 nm. Using pH vs. concentration calibration curves concentrations of unknown ascorbic acid solutions were determined.

### (b) Thermal Stability

Thermal stability was measured using a STA N-650 Simultaneous Thermal Analyzer operating in the differential scanning calorimetry (DSC) and thermogravimetric analysis (TGA) modes.

TGA measures the thermal stability of a material and its volatile components by monitoring the weight change during heating a sample in an inert atmosphere, such as helium or argon. A plot of mass as a function of temperature (a thermogram) of the sample provides both qualitative and quantitative information about the material. DSC measures heat flow of a material as a function of temperature or time and provides for determination of the temperature range of a phase transition and the enthalpy of the transition. DSC can also be used to monitor the energy released or absorbed via chemical reactions during the heating process of sample. Samples were finely ground until homogenous and typically for TGA and DSC measurements were made at a scan rate of 10° C. min⁻¹ and a temperature range of 25 to 800° C.

### (c) Ultraviolet and Visible Spectrometry

Ultraviolet (UV) and visible measurements of solutions were made in a double-beam Shimadzu UV-1601 UV-Visible Spectrophotometer using two light sources: a deuterium lamp for ultraviolet light and a tungsten-halogen lamp for visible light. The UV region was scanned from 190 to 400 nm, and the visible region from 400 to 800 nm using solution samples.

The intensity of light passing through a sample (I) was measured and compared to the intensity of light of the beam passing through the reference (Iₒ). The ratio I/Iₒ is called the transmittance (T). The absorbance, A, is based on the transmittance:

A=−log₁₀ T

The wavelength of maximum absorbance is a characteristic value, designated as λₘₐₓ. Quantitative analysis was done using calibration standards and measuring their absorbance values at λₘₐₓ.

### (d) Infrared Analysis

Infrared (IR) spectroscopy was used to quantify samples. Nicolet 6700 FT-IR spectrophotometer was used to measure absorbance or transmittance value of samples. All samples were finely ground and mixed with fused KBr to form pellets in the measurements. Typical parameters included: scan range of 400-4000 cm⁻¹; resolution 4.00 cm⁻¹; and the number of scans were 32.

### (e) Atomic Absorption

Atomic absorption spectroscopy was used for qualitative and quantitative analysis of metals in liquid samples and Buck Scientific 200A Atomic Absorption Spectrophotometer was used.

### (f) X-Ray Diffraction

Siemens X-Ray Diffractometer D5000 was used to obtain X-ray powder diffraction spectra. X-ray diffraction method is suited for characterization and identification of polycrystalline phases and obtaining the nanolayer spacing of calcium montmorillonite and LAA-montmorillonite chelates.

### (g) Particle Size Determination

Automated sieve machine with sieves of mesh size 150 μm, 150-125 μm, 125-63 μm and 63 μm was used. First, 100.00 g of calcium montmorillonite was weighed and placed in a sieve with mesh size 150 μm. Sieves of mesh sizes 150-125 μm, 125-63 μm and 63 μm were stacked on top of one another in order of increasing mesh size, starting with the smallest at the bottom and the 150 μm sieve was placed on the top. These sieves were placed in the automated sieve machine and when the calcium montmorillonite had been efficiently sieved, the amount of calcium montmorillonite present on each sieve was weighed.

### L-Ascorbic Acid and its Derivatives

Ascorbic acid is a water-soluble carboxylic acid of formula C₆H₈O₆ containing four hydroxyl groups in positions 2, 3, 5 and 6; the hydroxyl group in position 3 is acidic (pK_{a,3}=4.2), the hydroxyl in position 2 has pK_{a,2}=11.6, while hydroxyl groups in positions 5 and 6 behave as a secondary and primary alcoholic residue respectively. The structure of LAA is as shown below.

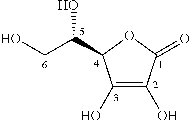

The structure of LAA derivative is as shown below:

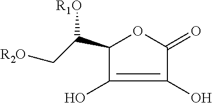

wherein R₁ and R₂ is independently selected from a member of the group consisting of linear or branched alkyl having 1 to 20 carbon atoms, an alkylcarbonylmethyl, an alkoxycarbonyl methyl, an alkyl carbonyl ethyl, an alkoxy carbonyl ethyl, an allylalkyl, an acyl, a sulphonic acid group an phosphoric acid group, and hydrogen, with the proviso that both R₁ and R₂ are not hydrogen.

Suitable L-alkyl-ascorbic acid derivatives include L-methylascorbic acid, L-ethylascorbic acid, L-propylascorbic acid, L-isopropylascorbic acid, L-butylascorbic acid, L-isobutylascorbic acid, L-pentylascorbic acid, L-hexylascorbic acid, L-octylascorbic acid, L-decylascorbic acid, L-dodecylascorbic s acid, L-tetradecylascorbic acid, L-hexadecylascorbic acid, L-octadecylascorbic acid and L-didecylascorbic acid. Suitable esters are L-ascorbic acid- phosphate ester and a salt thereof, L-ascorbic acid-sulfate ester, L-ascorbic acid-pyrophosphoric acid, L-stearylascorbic acid, L-palmitoylascorbic acid and L-dipalmitoylascorbic acid.

### Montmorillonite

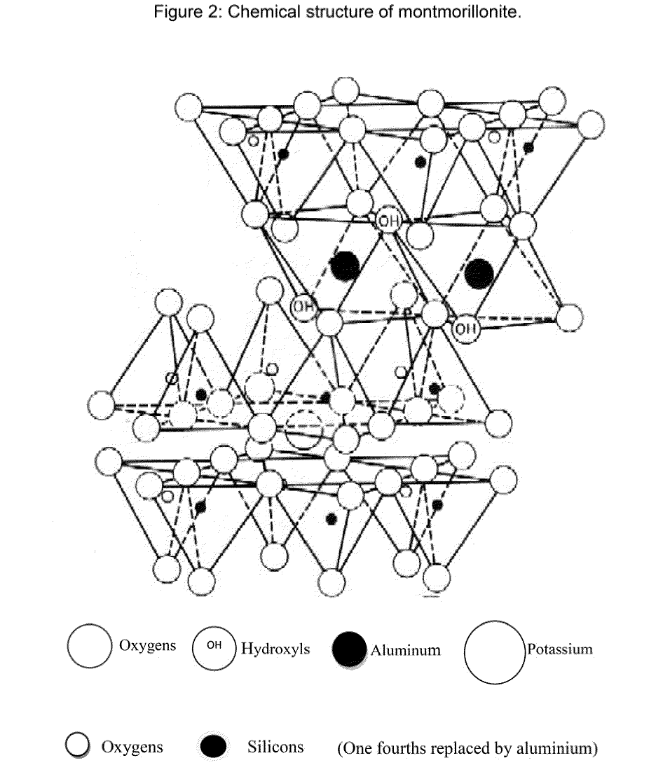

Montmorillonite is a soft phyllosilicate mineral of the smectic group and has the general formula (Na,Ca)_{0.33}(Al,Mg)₂Si₄O₁₀(OH)₂.nH₂O and its chemical structure is shown in FIG. 2. It typically forms microscopic, plate-shaped crystals that are extremely fine-grained and thin-layered compared to that of the other clay minerals. The interlayer spacing is expandable and typically varies from about 1.0 to about 2.0 nm. Theses interlayers are termed nanolayers and typically contain cations loosely bound to one another. An important property of the montmorillonite clay is the exchangeability of these cations present within these nanolayers. Montmorillonite has a higher cation exchange capacity than the simpler species of clay, such as kaolinite and this property is important when it is necessary to adsorb toxic metal ions in environmental cleanups etc. See Calcium Montmorillonite, (n.d.). Retrieved: Sep. 3, 2008; http://www.calearthminerals.com/aboutcalcium.php; Fullers Earth Clay Profile (n.d.). Retrieved: Sep. 3, 2008, http://www.mountainroseherbs.com/learn/fullers.php.

Typical values of the cation exchange capacity montmorillonite are 60-40 mol/kg and this high cation exchange capacity is useful when it is necessary to prepare montmorillonite containing specific type of ion within the nanolayers. In addition montmorillonite has a large surface area when hydrated in water boosts its adsorptive and absorptive properties. Toxins can stick to its outside surface and numerous elements and organic matter can enter the space between these nanolayers. Montmorillonite is important for detoxification purposes in the body because it contains high amounts of negatively charged ions. Since all toxins are positively charged the negative ions attract the toxins' positive ions, facilitating the movement of toxins through the kidneys or lymphatic system to a site of normal excretion of the toxins. Typically high surface area of montmorillonite (˜800 m²/g) allows the adsoption of positively charged toxins many times its own weight. Montmorillonite is also used in animal feeds as an anti-caking agent as it has the ability to bind mycotoxins in the digestive system of animals as well as several bacteria in vivo.

The common types of montmorillonite are sodium and calcium montmorillonite. Sodium montmorillonites are industrial clays typically used in plasters, oil-well drilling muds, soil additives and lubricating greases. Calcium montmorillonite is also known as “living clay” and is edible. Calcium montmorillonite contain trace mineral elements which are vital to the cellular functions of living creatures and that necessary for vitamins and enzymes to function.

### LAA-Calcium Montmorillonite Chelate.

X-ray diffraction analysis, IR analysis, TGA and DSC of LAA and calcium montmorillonite indicated that LAA-montmorillonite chelate was formed at pH values of about 5 to about 11. The maximum amount of chelation was determined to occur when the pH of the medium was about 9.

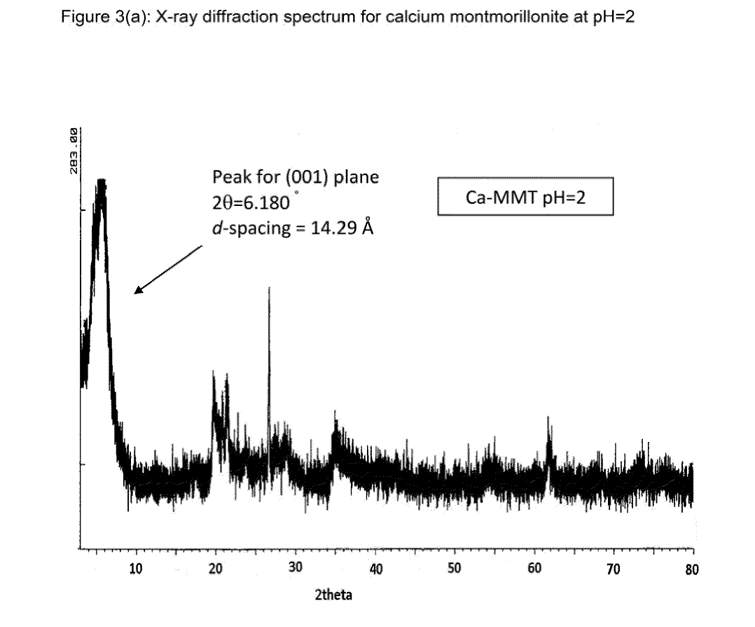

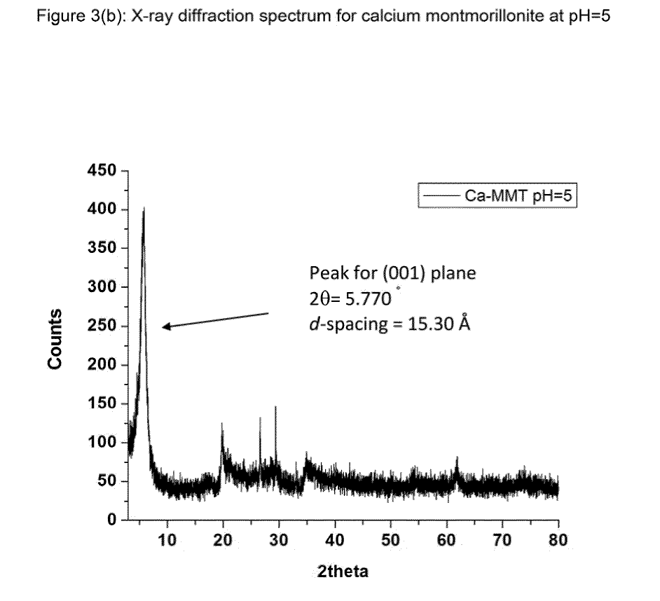

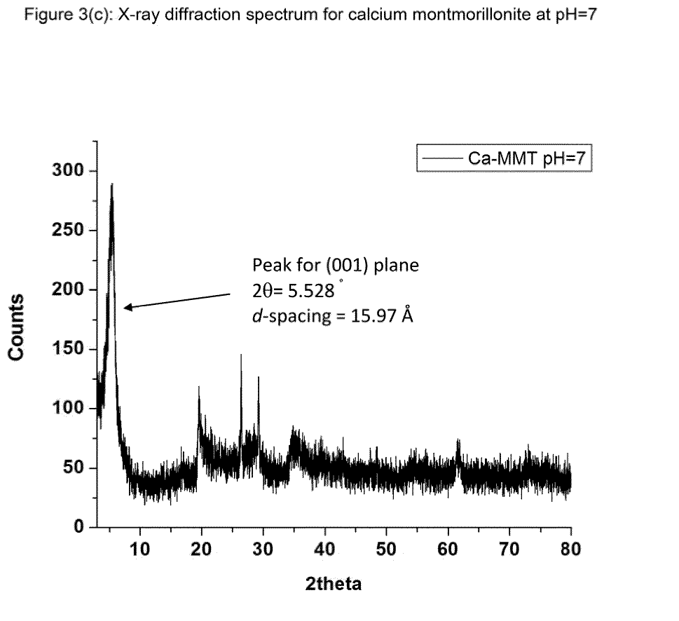

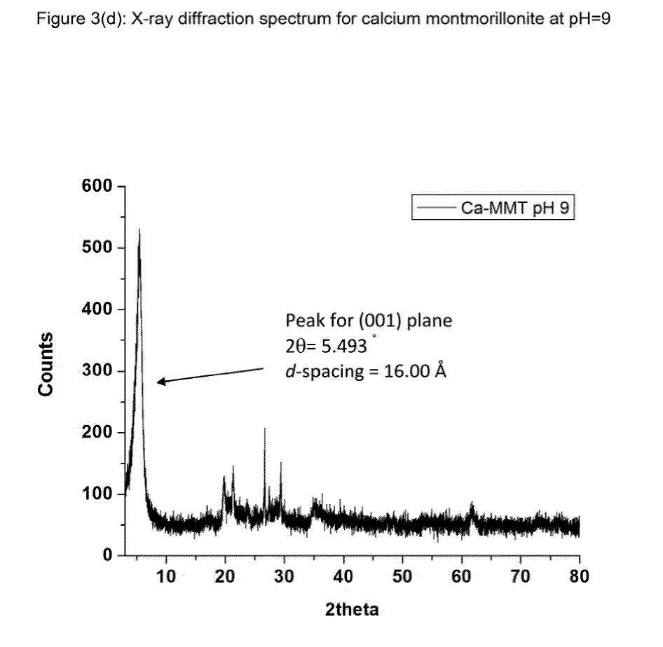

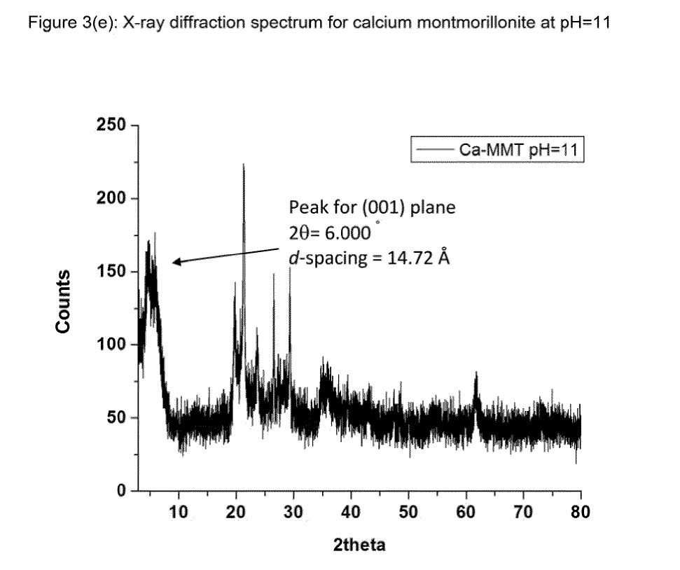

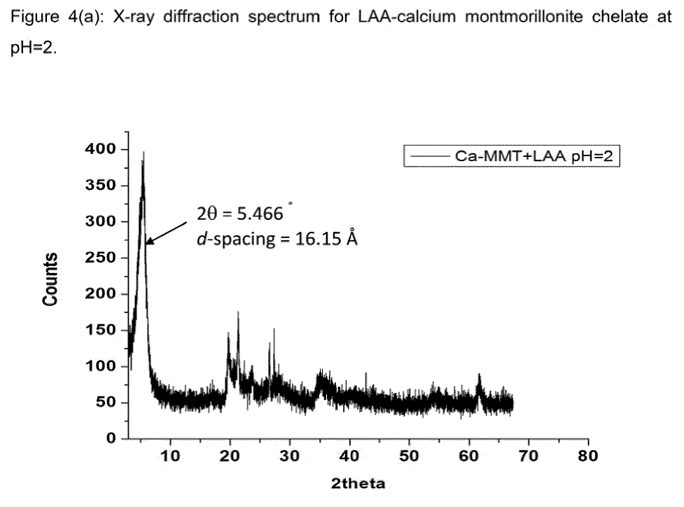

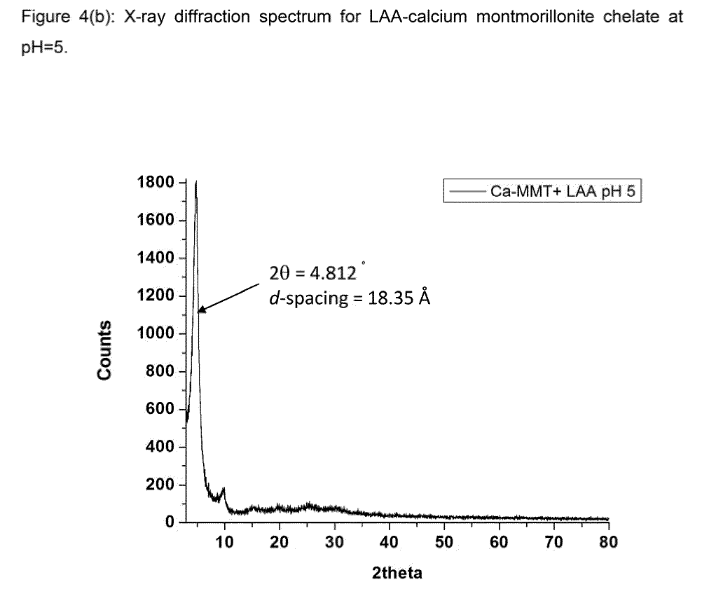

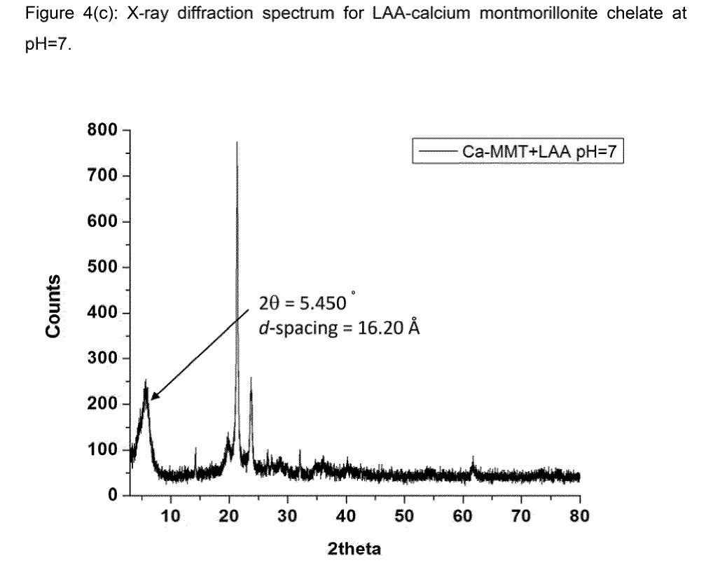

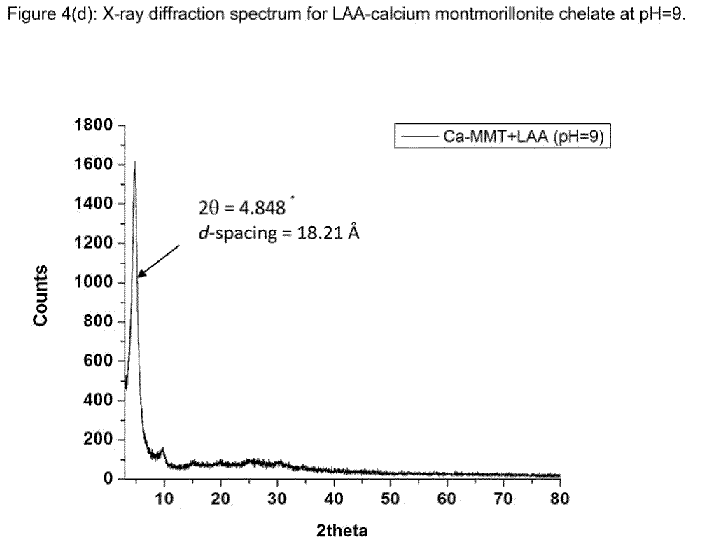

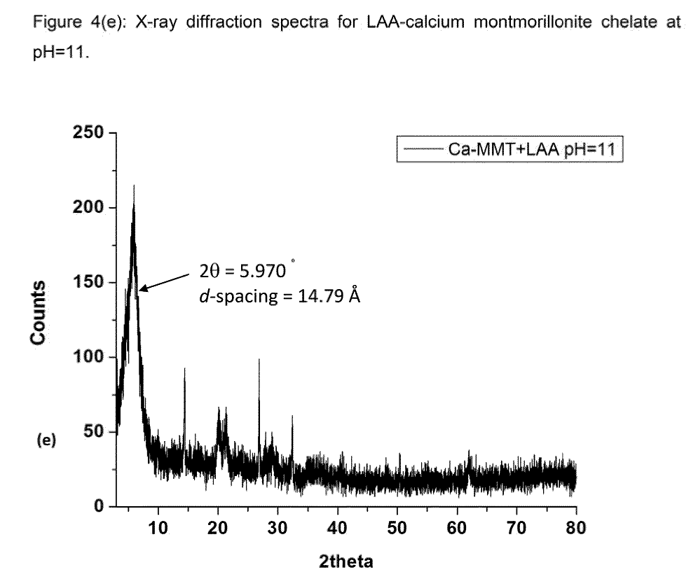

Nanolayer spacing of calcium montmorillonite and LAA-montmorillonite chelates were obtained by X-ray diffraction measurements. See, FIG. 3 and FIG. 4. The nanolayer spacing was found to increase as the pH was increased (see Table 1, first column) and reached a maximum at pH of 9.0. The nanolayer spacing decreased as the pH was increased to 11.0. At low pH values exchange of Ca²⁺ ions with H⁺ ions within the nanolayer can occur. Since H⁺ ions are smaller in size than Ca²⁺ ions, H⁺ ions can be enclosed in a hydration sheath. Accordingly, an increase in nanolayer spacing is expected with decreasing pH values. However, this is not the case. It is believed that H⁺ ions are not enclosed in a hydration sheath but forms hydrogen bonded H₃O⁺ structures as shown hereinbelow:

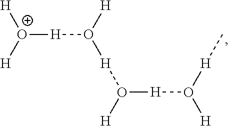

When pH is increased, the H⁺ concentration decreases and the exchange of Ca²⁺ to H⁺ decreases. This results in an increase in nanolayer spacing and the maximum nanolayer spacing is observed at pH about 9.0. When pH is increased to 11.0 the nanolayer spacing decreases and this can be explained by the availability of OH⁻ ions. It is belived that as the OH⁻ concentration increases (with increased pH) these OH⁻ ions diffuse into the nanolayer spacing and combines with H⁺ to form water structure as indicated above. This results in the decrease of nanolayer spacing at pH values greater than 9.

Referring to Table 1 second column, the nanolayer spacing of LAA-montmorillonite chelate increased for pH range 2 to 5 and pH range 7 to 9; maximum nanolayer spacing of 18.21 nm observed for pH value of 9. At a higher pH of 11.0 the nanolayer spacing decreased to 14.78 nm. At pH value of 2.0 the LAA is in its molecular form and the tendency for the molecular form to donate its lone pairs on the oxygen atoms and act as a ligand (or a counter-ion to Ca²⁺) is low. As the pH is increased from 5 to 9, LAA is deprotonated to gain a negative charge and the amount chelated within the nanolayer increased. When the pH reaches 11.0, the OH⁻ ion concentration is greatly increased than that of the ascorbate anion. The ascorbate ion competes with the OH⁻ ion to be incorporated in the interlayer spacing and leads to lower expansion of the nanolayer spacing.

TABLE 1
Nanolayer spacing of calcium montmorillonite chelated with LAA
(mass ratio 1:5) at different pH values.

| | nanolayer spacing before | nanolayer spacing after |
|---|---|---|
| pH | chelation/±0.01 Å | chelation/±0.01 Å |
| 2.0 | 14.29 | 16.15 |
| 5.0 | 15.30 | 18.35 |
| 7.0 | 15.97 | 16.20 |
| 9.0 | 16.00 | 18.21 |
| 11.0 | 14.72 | 14.79 |

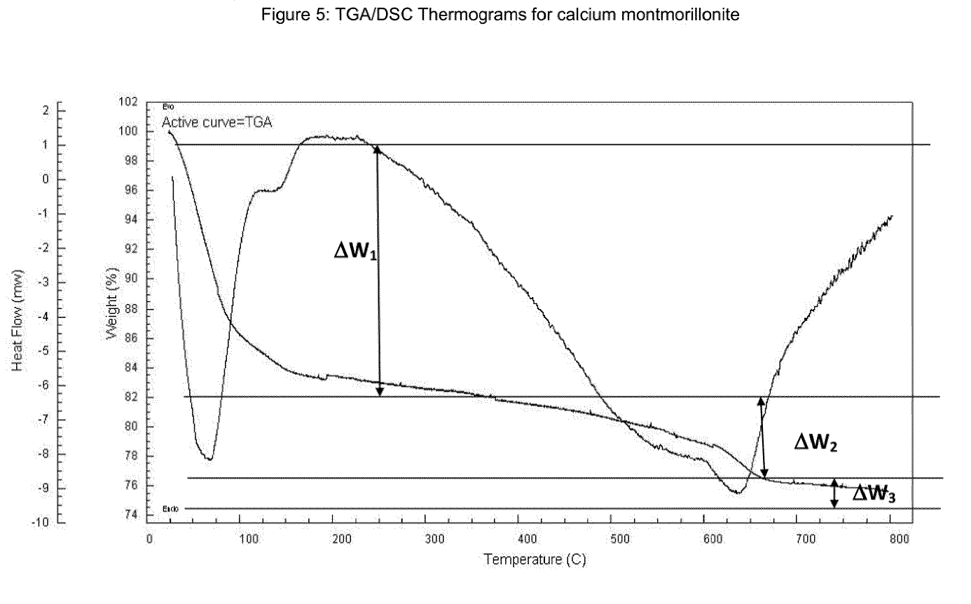

Referring to FIG. 5, the shoulder present in the TGA/DSC thermograms for calcium montmorillonite was assigned to the interaction of calcium ions with anions of montmorillonite. Three regions of weight loss were observed in the TGA plot: ΔW₁ is the weight loss occurring due to evaporation of free water, found in the nanolayers and occurs until about 100° C.; ΔW₂ is the weight loss due to evaporation of water molecules bound to the calcium cations present in the nanolayers; and ΔW₃ occurs from about 600° C. and higher due to loss of hydroxyl groups in the aluminosilicate structure and at this point the structure of the montmorillonite layers collapses.

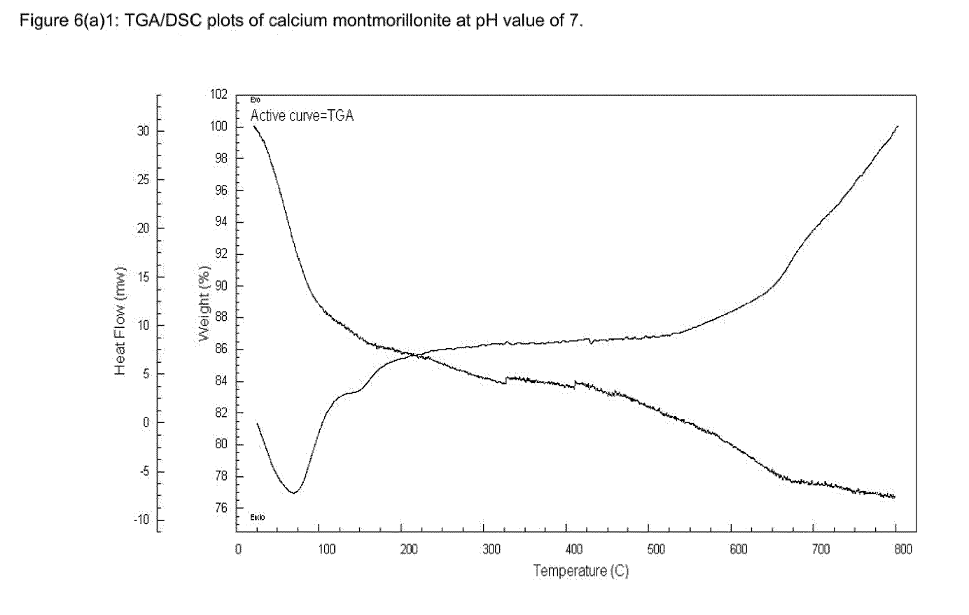

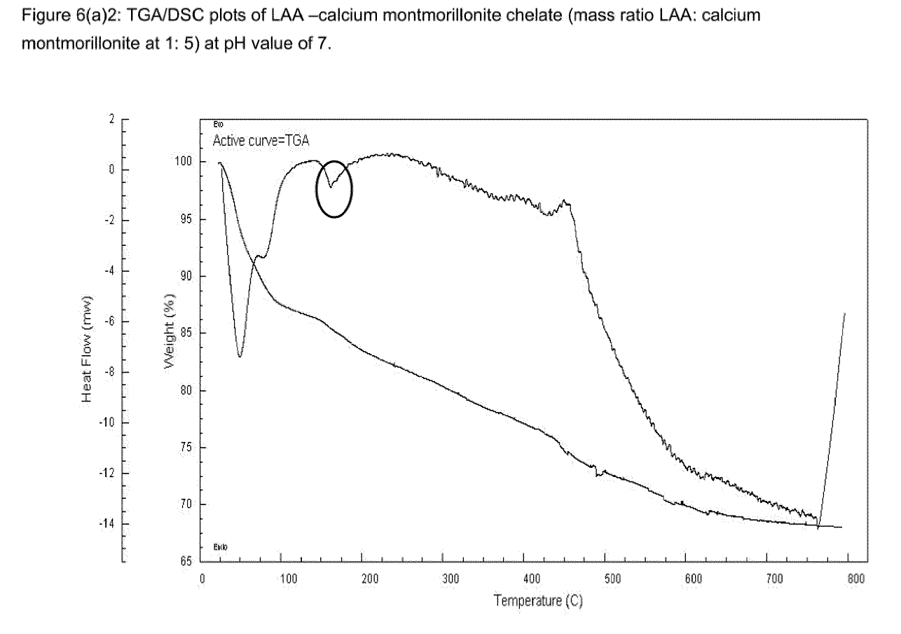

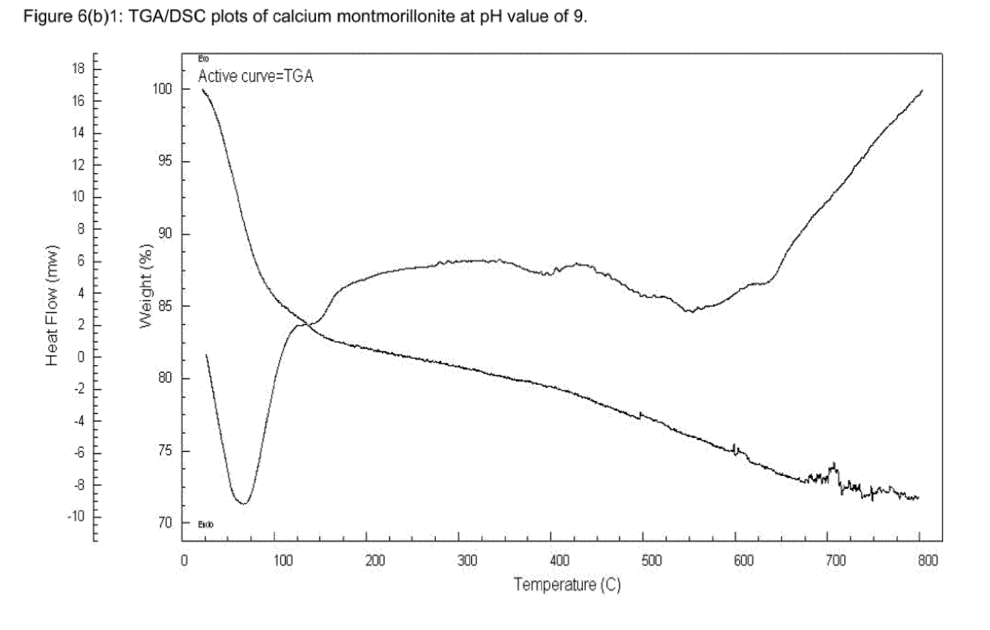

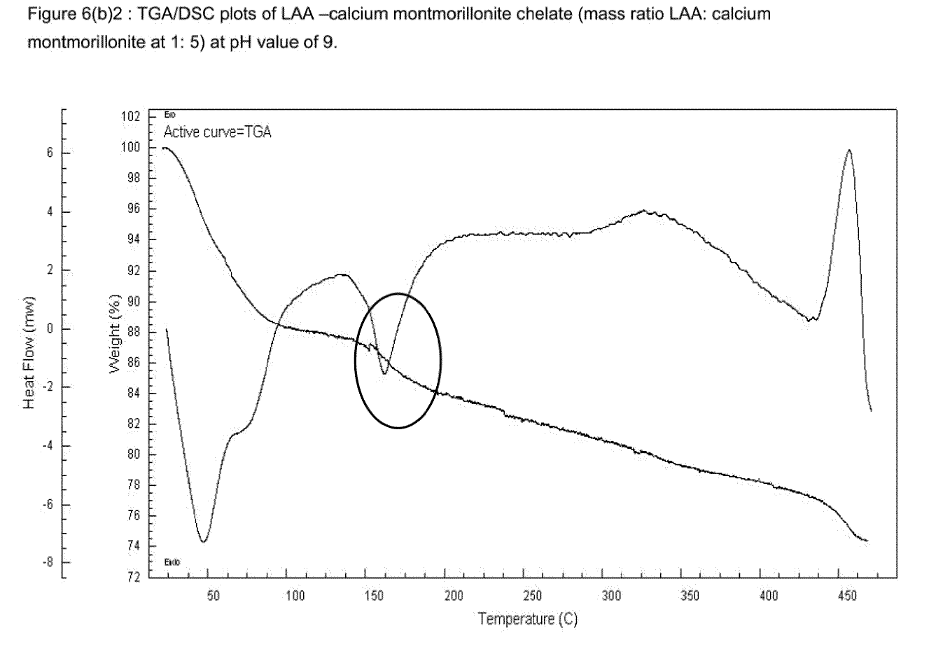

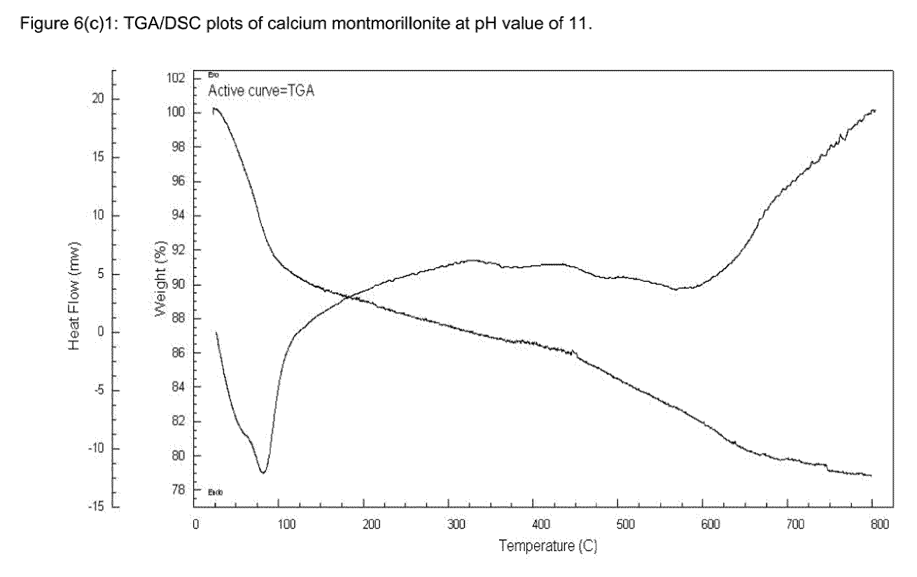

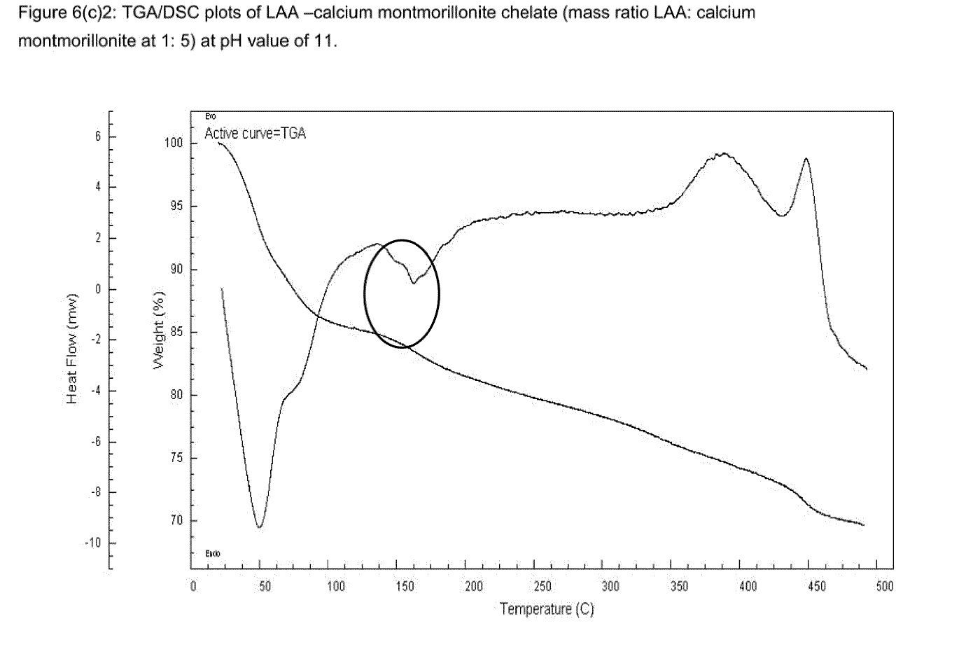

Referring to FIG. 6, in the TGA/DSC plots of the LAA-calcium montmorillonite chelate, a trough was observed between 150 to 200° C. in the DSC thermogram. This corresponds to melting of LAA as the melting point range of LAA acid is 190 to 192° C. A maximum of the trough was seen at pH of about 9.0 where most of the LAA was chelated.

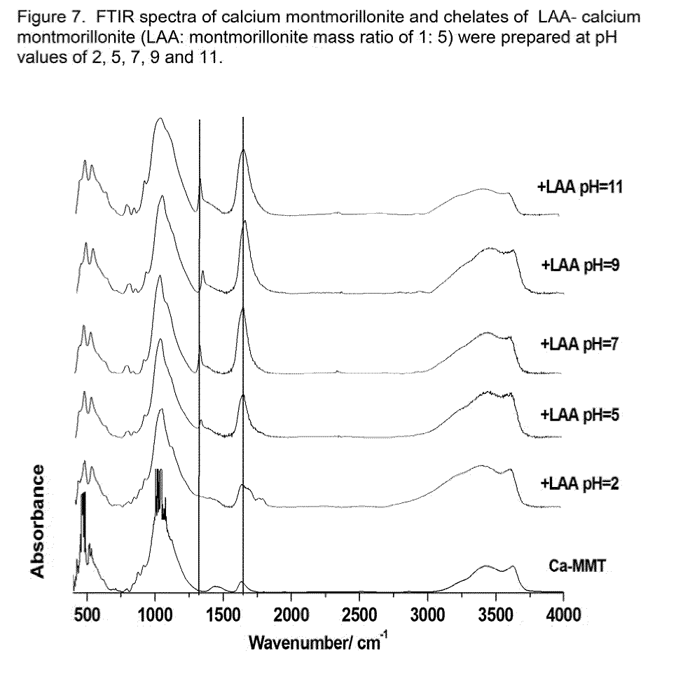

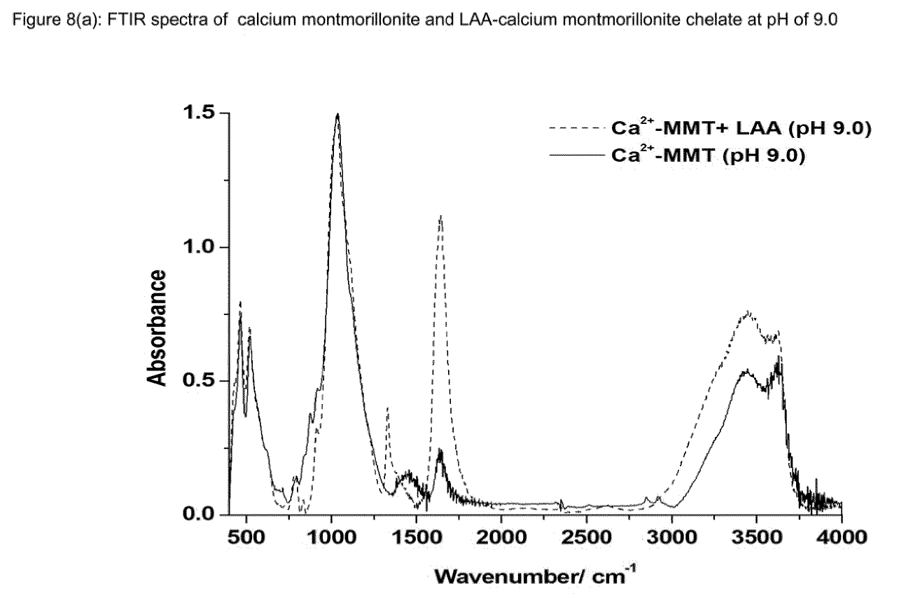

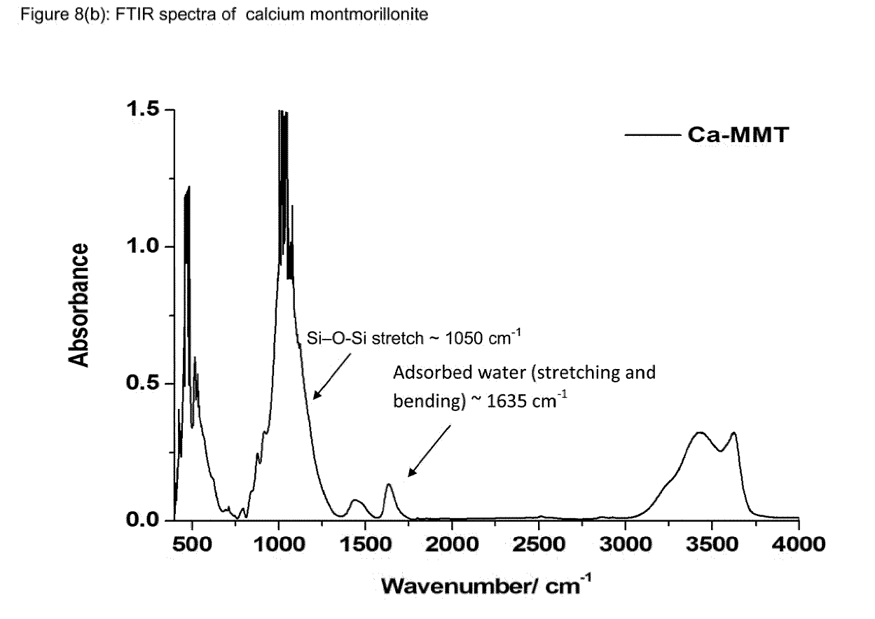

Referring to FIG. 7, the FTIR spectra of LAA-calcium montmorillonite chelate indicated that peaks for stretching and bending of adsorbed water on montmorillonite (˜1635 cm⁻¹) overlaps with the C═C stretch (˜1630 cm⁻¹) of LAA. The peak between 1250-1500 cm⁻¹ was present in the spectra of the LAA-calcium montmorillonite chelate, but not found in the calcium montmorillonite spectrum. This was assigned to the asymmetric C—O stretch of LAA. The change in the broad band at 3400-3600 cm⁻¹ was attributed to the hydrogen bonded Al—OH stretch of montmorillonite. The intensity of this band was increased because overlapping of the —OH stretches of LAA. By comparing the spectra it was observed this band increases as the pH of the media was increased during preparation of the LAA-calcium montmorillonite chelate. This leads to the inference that more ascorbic acid is chelated at higher pH values. These observations were consistent with TGA/DSC results and X-ray diffraction results. See FIG. 8 for FTIR spectra of calcium montmorillonite and LAA-calcium montmorillonite chelate that were prepared at pH of about 9.0.

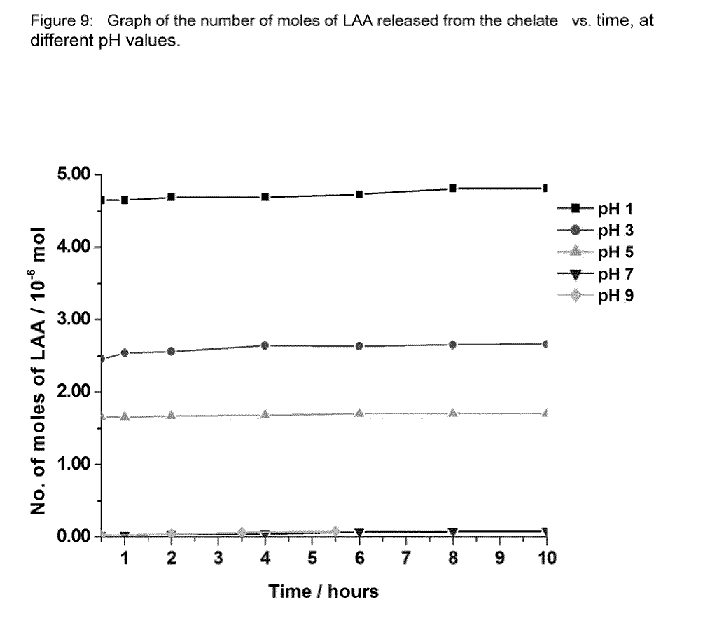

In FIG. 9, comparison of the released number of moles of LAA at the different pH values is presented. It was observed that the amount of LAA released drops significantly as the pH of the surrounding solution was increased. At high concentrations H⁺ ions diffuse into the nanolayers of montmorillonite and recombines with the ascorbate ion. This results in the release of calcium cations from the LAA-calcium montmorillonite chelate. As seen from the reaction hereinbelow the equilibrium is shifted to the left and according to Le Châtelier principle an increase in LAA concentration is obtained. Second deprotonation reaction of LAA is believed to be negligible because the high pKa (11.6) of deprotonation of the second proton.

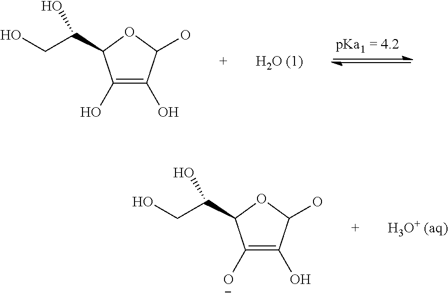

As the pH is increased by introduction of OH⁻ ions into the medium, the hereinabove equilibrium is shifted towards the right and the ascorbate forms the stable chelate with calcium cation within the nanolayer.

### Thermal Stability of the LAA-Calcium Montmorillonite Chelate

Thermal stability of LAA was increased significantly when it was chelated within the monolayer of calcium montmorillonite. The thermal stability at 200° C. is greater than the melting point range (190-192° C.) of LAA and this stability is attributed to the strong chelating bonds formed between calcium cations and the ascorbic acid. Higher thermal energy is required to break the chelate structure. At pH of about 9, more than 95% of the LAA is chelated. In an embodiment more than 98% of LAA is believed to be chelated.

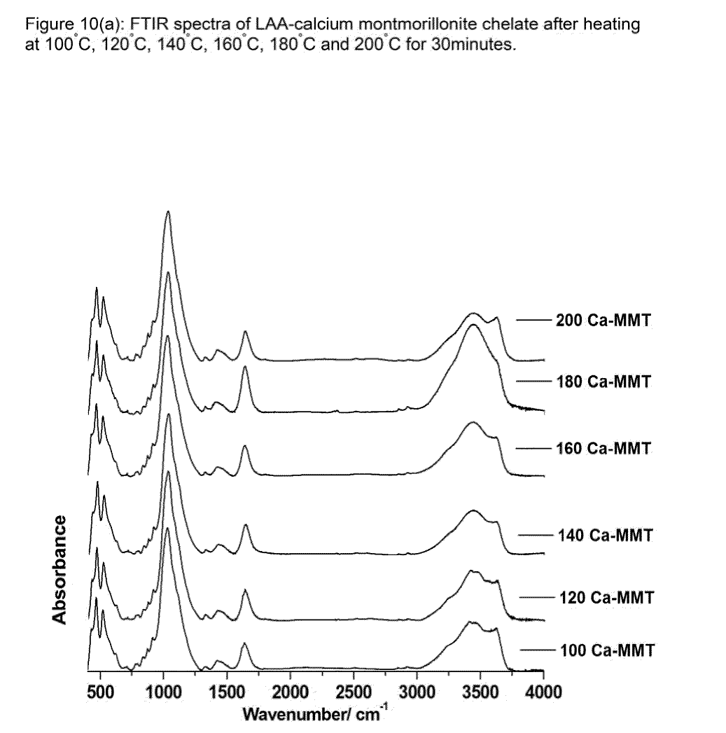

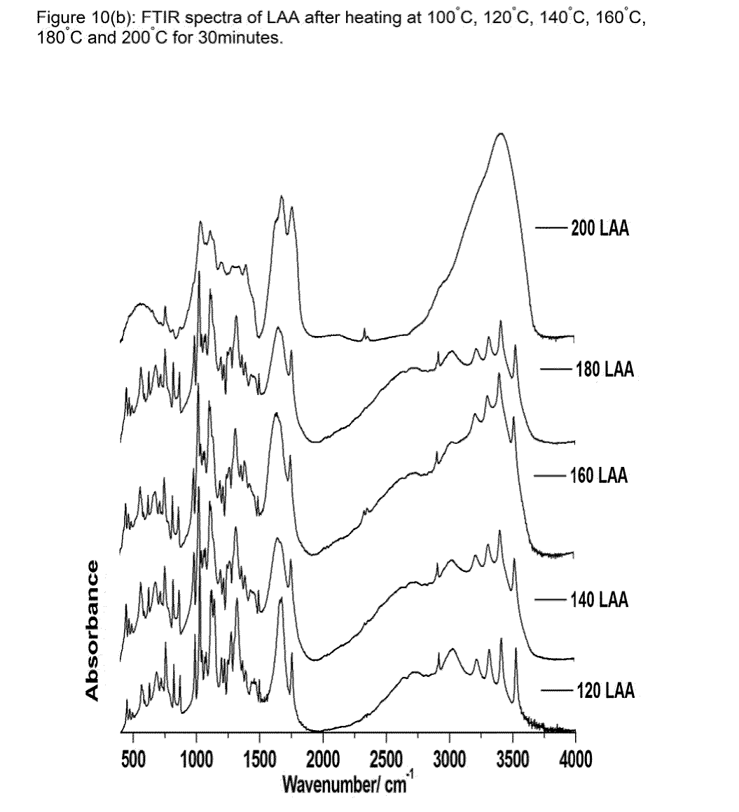

The FTIR spectra of the 0.5000 g calcium montmorillonite chelated with 0.2500 g of LAA heated at 100° C., 120° C., 140° C., 160° C., 180° C. and 200° C. for 30 minutes are shown in FIG. 10. As reference, the FTIR spectra of LAA at room temperature and after heating to 90, 120, 140, 160, 180 and 200° C. for 30 minutes are also shown in FIG. 10.

As seen from the FTIR spectra of FIG. 10, the structure of LAA-calcium montmorillonite chelate does not change significantly as the temperature is increased, (except for the case where it was heated to 180° C.). Examination of the fingerprint regions indicate that the structure of pure LAA changes gradually as the temperature is increased until it is completely destroyed. Further at 140° C., the structure of LAA is altered as the carbonyl stretch at 1750 cm⁻¹ was broader and the fingerprint region was changed. The FTIR spectra of LAA before heating and after being subject to heating at 90° C. for 30 minutes is shown in FIG. 11.

### Vitamin C Supplements

Manufacture of Vitamin C supplements typically requires addition of binders such as starch, sucrose and fillers such as talc. Any other pharmaceutically acceptable additives or excipients can be used in the manufacture of these supplements. For example, in pellet manufacture at least 25% of fillers are used. Additionally, excipients such as fillers added to ascorbic acid can cause the ascorbic acid to oxidize. Moreover, fillers may be detrimental to overall health in the long term and may, in themselves, cause side effects. Thus, it is desirable to manufacture a Vitamin C supplement that is administered in a pellet form which contains a minimum amount of additional substances. Further application of heat during pellet manufacture also leads to oxidation of Vitamin C. Even though supplements are an important source of Vitamin C to the body, it is important to be mindful of the dosage limit, as an excess can lead to damaging side-effects. The Food Standard Agency (UK) recommends 100 mg/day, which is just above the Reference (or Recommended) Daily Intake (RDI) for Vitamin C of 90 mg/day. See MCacleod J. (ed), 1968, Davidson's Principles and Practice of Medicine, 14^{th} edition, Churchill Livingstone, Great Britain.

Excess accumulation of Vitamin C is avoided in the body in at least three ways. First, amount of Vitamin C absorbed in the gut reaches a maximum at relatively low doses. Virtually all the Vitamin C that is absorbed from the gut is thus excreted in the urine. Second, the kidney rapidly excretes vitamin C. Third, tissue uptake of Vitamin C is also saturable. Vitamin C is partly excreted as oxalate, and very high doses can lead to hyperoxaluria and kidney stones, particularly after intravenous use and in persons with renal insufficiency. Adverse effects of Vitamin C absorbed in the gut include nausea, abdominal cramps and diarrhea.

It is well recognized that sustained release or controlled release tablets or pellets are formulated so that the active ingredient is embedded in a matrix of insoluble substance. This allows the dissolving drug to find its way out through the holes in the matrix, and is therefore released over a period of time. Advantages of sustained or controlled release formulations are that they can often be taken less frequently than instant-release formulations of the same drug. Further, steadier levels of the drug are maintained in the bloodstream. An advantage of a controlled or sustained release formulation for LAA is that a continuous supply of LAA is maintained. Typically the biological half-life for Vitamin C is fairly short, about 30 minutes in blood plasma. Since pharmacological activity of Vitamin C is related to its level in the blood time releasing is important. A benefit of using calcium montmorillonite in controlled release or sustained release formulation is that since Ca²⁺ is surrounded by an organic molecule provides for increased absorption of calcium. Accordingly, this is a viable source of calcium supply to the body.

According to a study done by Lee et al., dose-response analysis revealed that once montmorillonite was combined with L-ascorbic acid, the EC₅₀ of montmorillonite intercallated with LAA was significantly larger than that of montmorillonite and L-ascorbic acid implying that montmorillonite-LAA was much less toxic than LAA and montmorillonite. See Y. H. Lee, T. F. Kuo, B. Y. Chen, Y. K. Feng, Y. R. Wen, W. C. Lin , F. H. Lin, 2005, Toxicity Assessment of Montmorillonite as a Drug Carrier for Pharmaceutical Applications: Yeast and Rats Model, Biomedical Engineering Applications, Basis and Communications, vol. 17, no, 2, pages 12-18.

### Human Gastrointestinal Tract and Drug Absorption

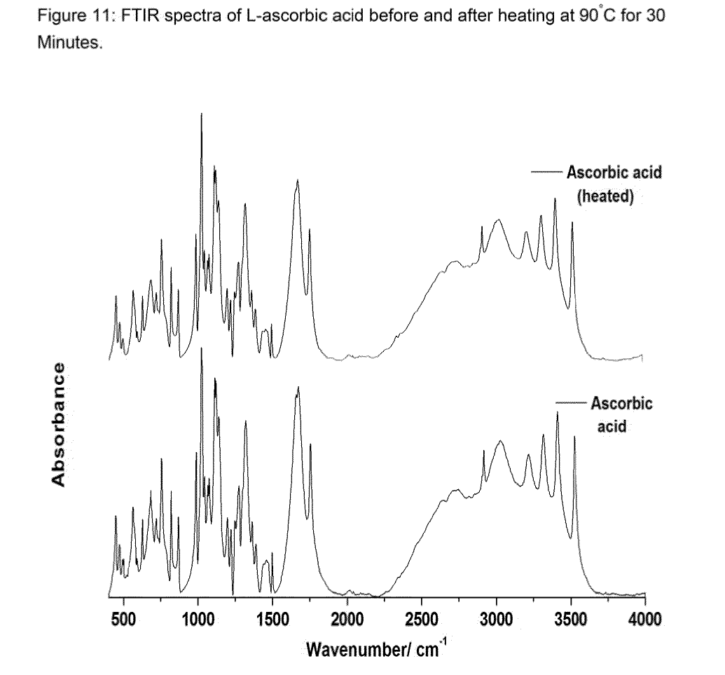

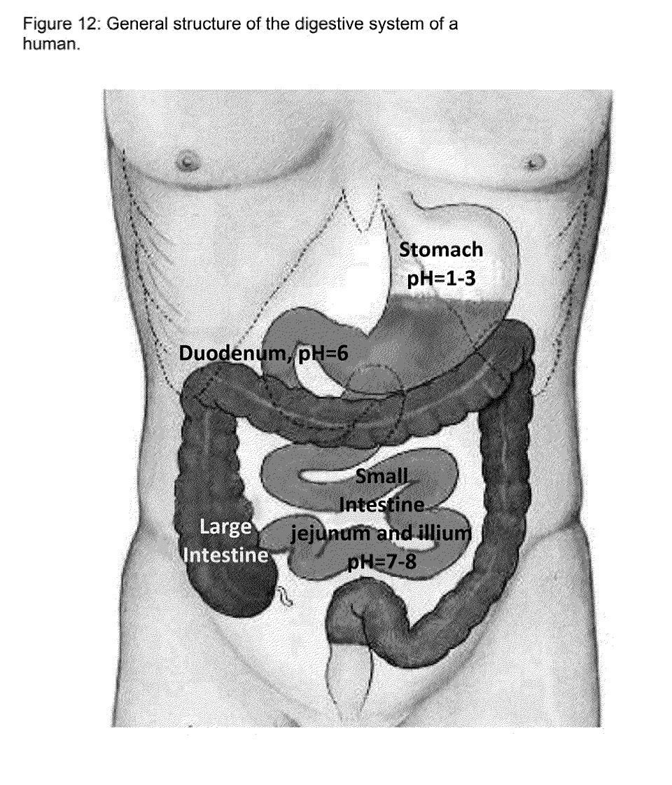

A schematic diagram of the human gastrointestinal tract (GI) is shown in FIG. 12. The GI tract is typically about 6.5 meters long and can be divided into portions based on the organs of digestion along the alimentary canal. Each portion along the digestive tract has a different pH value. Usually the tablet, capsule, solution or suspension administered orally passes quickly to the stomach, via the esophagus. Drug absorption does not normally occur in the esophagus, because the transport time is rapid.

Stomach contents of a human include hydrochloric acid, pepsinogen, and mucus and the pH of the stomach varies within the pH 1-5 range. As stomach is an organ of digestion and very little absorption occurs except for water, ions and drugs such as aspirin. Along the alimentary canal is the small intestine. The small intestine consists of duodenum and the pH of the duodenum is about 6 to 6.5. The majority of nutrients, vitamins, and drugs are absorbed in this 6 inch area of the GI tract. The lining of the small intestines is composed of many villi, or finger like projections consisting of several thousand projections termed the brush border. This whole area is highly perfused with blood and provides a very large surface area through which absorption can occur efficiently.

Beyond the duodenum lies the jejunum and ileum. These sections of the small intestine lack the high surface area of the duodenum and only small amounts of absorption of vitamins occur across lipid membranes. The pH rises to about 7.5 in this region. The final organ of the digestive tract is the large intestine, which includes the colon and rectum. The large intestine is the site for water resorption and the production of faeces. Generally, drug absorption does not take place in this region. The pH of the large intestine ranges from about 5 to 7.

Food remains in the stomach, on an average from 2 to 4 hours, depending on the volume and type of food. After food in the stomach has become thoroughly mixed with the stomach secretions, the resulting mixture that flows through the gut is called chyme. About 3 to 5 hours are required for passage of the chyme through the small intestine, until it is emptied at the ileocecal valve into the large intestine. Typically, 8 to 15 hours is sufficient to transport the chyme through the colon and exit the body. See Guyton A. C., Hall J. E., 2006, Textbook of Medical Physiology, 11^{th} edition, Elsevier Inc., Philadelphia.

### Gastrointestinal Tract and Absorption of Vitamin C

The GI absorption of ascorbic acid occurs through an active transport process, as well as through passive diffusion. At low gastrointestinal concentrations of ascorbic acid active transport predominates, while at high gastrointestinal concentrations active transport becomes saturated, leaving only passive diffusion. Slowing down the rate of gastric emptying (for example, by taking Vitamin C with food or taking a slow-release formulation usually increases absorbed amounts.

For vitamins to be absorbed through the walls of the GI tract, it is necessary that the vitamin to be in its neutral, molecular form so that it can diffuse through the lipid layer of the tissue. LAA is an acidic molecule that ionizes in the presence of basic conditions or high pH values. Absorption of LAA is believed to take place from the GI tract in the regions where the pH is acidic, that is, in the stomach and in the duodenum.

Leaching of LAA is dependent on the amount of LAA present in the s montmorillonite chelate with the highest leaching rate observed occurs within the first couple of hours and the lowest leaching rate occurring around the 8^{th} to the 10^{th} hour. With time LAA accumulates in the aqueous solution and the concentration gradient decreases. This suggests that release of LAA can occur at a low pH at gastric pH ranges of 1-3. When ingested the released calcium ascorbate from the chelate can be hydrolyzed in the stomach to give calcium and L-ascorbate ions at a stomach pH of 1-3. The acid in the stomach (HCl) converts the L-ascorbate ions to LAA giving rise larger ingested amounts without the increase of stomach acid concentration without risk of acid upset or diarrhoea. This suggests that the LAA-montmorillonite chelate can be a source of a calcium supplement as well as being a Vitamin C supplement.

Further, as the amount leaching of LAA drops significantly as the pH of the surrounding solution is increased to about 6 suggesting that lower leaching occurs in the dudendum. Also, the chelation of L-ascorbate ions with calcium cations protects it from oxidation to L-dehydroascorbic acid and subsequent rapid oxidative degradation in the duodenum in the pH region around 6 that is present in the duodenum.

### Digestive System of the Chicken

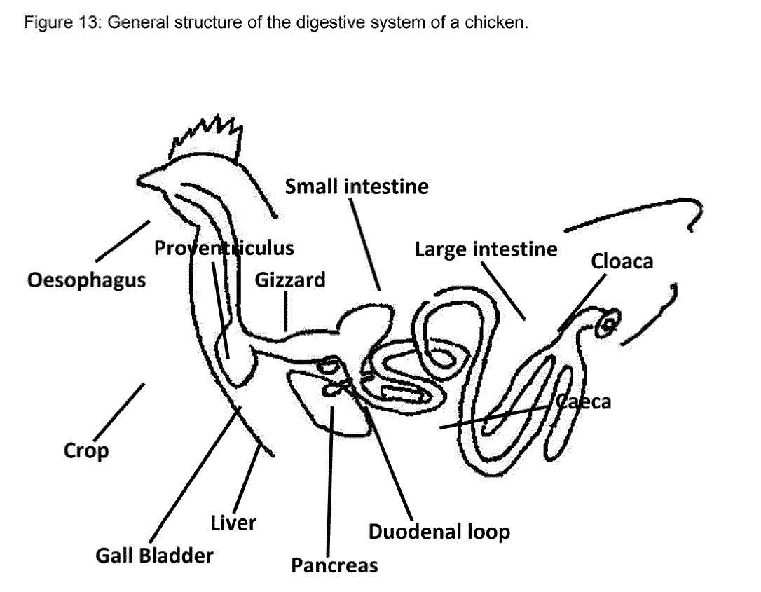

The digestive system of the chicken begins at the mouth and ends at the cloaca, as shown in FIG. 13. Intervening organs/parts include the oesophagus, crop, proventriculus, gizzard, duodenum, small intestine, paired caeca and large intestine. The oesophagus in a chicken is a flexible tube which carries food from the mouth to the crop. A temporary storage pouch located at the base of the neck sends the hunger signal to the chicken brain. The oesophagus which traverses the chest cavity, carries food to the proventriculus where the food is mixed with acids (HCl) and other digestive enzymes. Grit in the gizzard, combined with strong muscular action, grinds the food into a mash.

The small intestine starts at the exit of the gizzard. Food in the duodenum is neutralised by the addition of more enzymes excreted by the pancreas and these enzymes break down proteins. The products of digestion are absorbed from the small intestine and carried to the liver.

The pH of the contents of the chicken digestive tract is shown in Table 2. See Whittow G. C, 1976, Sturkie's Arian Physiology, 5^{th} edition, Academic Press, USA.

TABLE 2
pH of the contents of the chicken digestive tract

| Crop | Proventriculus | Gizzard | Duodenum | Jejunum |
|---|---|---|---|---|
| 4.51^{a} | 4.8^{a} | 4.74^{c} | 5.7-6.0^{a} | 5.8-5.9^{a} |
| | | 2.50^{a} | 6.4^{c} | 6.6^{c} |

| Ileum | Rectum | Caeca | Mouth |
|---|---|---|---|
| 6.3-6.4^{a} | 6.3^{a} | 5.7^{a} | 6.7^{b} |
| 7.2^{c} | | 6.9^{c} | |
| | | 5.5-7.0^{d} | |

^{a}Whittow G. C, 1976, Sturkie's Arian Physiology, 5^{th} edition, Academic Press, USA.
^{b}Herpol, C. (1966) Influence de l'ago sur le pH dans le tube digestif de gallus domesticus. Ann. Biol. Anim. Biochim. Biophys. 4, 239-244
^{c}Herpol, C. and van Grembergen, G. (1967) La signification du pH dans le tube digestif de gallus domesticus. Ann. Biol. Anim. Biochim. Biophys. 7, 33-38
^{d}Sudo, S. Z. and Duke, G. E. (1980) Kinetics of absorption of volatile fatty acids from the ceca of domestic turkeys. Comp. Biochem. Physiol. A67, 231-237
^{e}Lin, G. L., Himes, J. A. and Cornelius, C. E. (1974) Bilirubin and biliverdin excretion by the chicken. Am. J. Physiol. 226, 881-885

An example of variation of transit timein the chicken broilers is shown in Table 3 hereinbelow. See, Whittow G. C, 1976, Sturkie's Arian Physiology, 5^{th} edition, Academic Press, USA.

TABLE 3
Retention time of food in chicken broilers

| Organ | Time/minutes |
|---|---|
| Crop | 31 |
| Proventriculus and gizzard | 39 |
| Duodenum | 10 |
| Jejunum | 84 |
| Ileum | 97 |
| Caeca | 119 |
| Rectum | 56 |

The release of Vitamin C at acidic pH values in the LAA-calcium montmorilonite is also compatible with the absorption areas in the GI tract of the chicken (small intestine) as the pH ranges from about 5.5 to about 7.0. These results suggest that LAA-montmorillonite chelate is suitable as a vitamin C carrier for chicken feeds.

The following non limiting examples are shown hereinbelow.

### EXAMPLES

### Example 1

### Preparation of Calcium Montmorillonite by Ca²⁺ Exchange of Montmorillonite

This was done to obtain calcium montmorillonite that was used in Examples 2-6.

First, 600.00 cm³ of a CaCl₂ (aq) solution of 1.000 mol dm⁻³ concentration was prepared by dissolving 66.6000 g solid CaCl₂ with 600.00 cm³ distilled water. 10.0000 g of montmorillonite was mixed into this solution stirred using a magnetic stirrer for 24 hours at room temperature and a suspension was formed. Next, the suspension was left to settle and the supernatant was decanted and discarded. The remaining precipitate was mixed with 600 cm³ of distilled water, shaken well and left to settle. The supernatant was decanted and tested with AgNO₃(aq) to establish whether Cl⁻(aq) ions were present and tested with H₂C₂O₄(aq) to check whether Ca²⁺(aq) ions were present. If present Ca²⁺ (aq) and ions were Cl⁻(aq) ions present, the precipitate was washed with distilled water again, in the manner described above, until no or very little Cl⁻(aq) and Ca²⁺(aq) remained. This washed precipitate was spread out on a watch glass and allowed to air-dry, at room temperature, until all moisture was removed. Finally, the dry calcium montmorillonite was ground to a powder, placed in a dessicator, and was used in Examples 2 through 6. The calcium montmorillonite used in these examples were assumed to be free of any surface-adsorbed Ca²⁺ as Ca²⁺ ions were not detected in the solution.

### Example 2

### Determination of Change in Nanolayer Spacing of Calcium Montmorillonite when the pH is Varied

First, a 0.5000 g sample of calcium montmorillonite prepared according to the procedure in Example 1 was weighed out and placed in a 100.00 cm³ beaker. Approximately 45 cm³ of distilled water was added to this, stirred and the pH was adjusted to pH of 2.0 using conc. HCl (aq). The final volume was made up to 50.00 cm³ with distilled water and the resulting suspension was stirred using a magnetic stirrer for 48 hours at room temperature. Afterwards, the suspension was centrifuged at 6000 rpm for 7 minutes and the precipitate obtained was spread out on a watch glass to air-dry at room temperature. After drying, the precipitate was powdered and the sample was subjected to X-ray diffractometry. Thermal gravimetric analysis was also carried out on a sample.

The procedure as described above was repeated to prepare samples at pH 5.0, 7.0, 9.0, 11.0, using conc. HCl and conc. NaOH (aq) solutions as necessary to adjust the pH. The calculated nanolayer spacing of the nanolayers from X-ray diffraction measurements are shown in Table 1 first column.

### Example 3

### Determination of the Maximum pH for Chelation of LAA with Calcium Montmorillonite

First, 5.0000 g solid LAA was weighed and placed in a 100.00 cm³ volumetric flask. Approximately 90 cm³ of distilled water was added to this and stirred well to dissolve the solid LAA. Then, 1.0000 g of calcium montmorillonite prepared according Example 1 was added to LAA solution and the pH was adjusted to pH of 2.0 using conc. HCl (aq). The final volume was made up to 100.00 cm³ with distilled water and was stirred using a magnetic stirrer for 24 hours at room temperature to form a suspension. Afterwards, the suspension was centrifuged at 6000 rpm for 8 minutes and the precipitate obtained was spread out on a watch glass to air-dry at room temperature. After drying, the precipitate was powdered and samples were subjected to X-ray diffractometry, infrared spectroscopy and TGA/DSC. A reference pellet containing pure LAA acid was also subject to infrared spectroscopy. The procedure was repeated to prepare samples at pH 5.0, 7.0, 9.0, 11.0, using conc. HCl and conc. NaOH (aq) solutions as necessary to adjust the pH. Comparison of the IR spectra and TGA/DSC plots indicated that at pH=9 the maximum chelate formation was obtained.

### Example 4

### Determination of the Maximum Amount of LAA that can be Chelated in Calcium Montmorillonite at the Previously Determined Maximum pH of About 9

First, 0.0500 g solid LAA was weighed out and placed in a 50.00 cm³ volumetric flask. Approximately 40 cm³ of distilled water was added and stirred well to dissolve the solid LAA. Then, 0.5000 g calcium montmorillonite was added to this solution, mixed using a glass rod and the pH was adjusted to pH value of 9.0 using conc. NaOH (aq). The final volume was made up to 50.00 cm³ with distilled water and was stirred using a magnetic stirrer for 24 hours at room temperature. Afterwards, the suspension was centrifuged at 6000 rpm for 8 minutes and the precipitate obtained was spread out on a watch glass to air-dry at room temperature. After drying, samples of the powdered precipitate were ground with fused solid KBr to form pellets and were subject to infrared spectroscopy. Next, the supernatant was centrifuged at 11500 rpm for 10 minutes and 1.00 cm³ portion of the supernatant obtained after centrifugation was titrated with a 0.005 mol dm⁻³ I₂/KI (aq) standard solution, to determine the amount of ascorbic acid remaining in the supernatant. The procedure was repeated using 0.1000 g, 0.2500 g and 0.5000 g of LAA, except that in these cases, 5.00 cm³ portions of the supernatant were used for titration with 0.005 mol dm⁻³ I₂/KI (aq). The results of the titration of the supernatants with I₂/KI (aq) are shown in Table 4.

TABLE 4
Average volume of 0.0005 mol dm⁻³l₂/Kl (aq) needed for titration and
amounts of LAA chelated

| Sample | Average volume of | Calculated | |
|---|---|---|---|
| with Ca²⁺- | 0.0005 mol dm⁻³ | mass of LAA | Mass of LAA |
| MMT:LAA | l₂/Kl (aq) | left in | chelated in 0.5 g |
| ratio | needed/±0.05 cm³ | supernatant/g | Ca²⁺-MMT/g |
| 10:1 | 0.50 | 0.004 | 0.046 |
| 5:1 | 4.17 | 0.037 | 0.063 |
| 2:1 | 16.88 | 0.149 | 0.101 |
| 1:1 | 10.85 | 0.477 | 0.022 |

The results from titration of the supernatants with I₂/KI (aq) show that the amount of LAA that was increased until the montmorillonite and LAA are in a mass ratio of 2:1, and the amount LAA was decreased. This observation can be explained by considering the effect of the increasing LAA concentration on the Ca²⁺ ions in the nanolayer spacing. At pH of 9.0, any ascorbic acid present is found principally in its deprotonated form. Therefore, as the mass of ascorbic acid applied is increased, the concentration of ascorbate ions in the aqueous solution increases. Also, to bring the pH to 9.0, higher amounts of NaOH (aq) is needed when more ascorbic acid is used. This means that the concentration of Na⁺ ions in the solution is also higher in the solutions containing more ascorbic acid. Due to the concentration gradients of Ca²⁺ and Na⁺ between the inter-layers and the aqueous surrounding, there is an exchange of cations. Ca²⁺ moves out of the layers while an equivalent number Na⁺ move into neutralize the negative charge of the layers (twice the number of Ca²⁺ ions). The diffusion of Ca²⁺ ions into the solution is also favored, because it forms a stable chelate with L-ascorbate present in the solution. The results of the atomic absorption tests performed on supernatants confirmed the presence of Ca²⁺ ions, validating the above explanation.

It was observed that the color of the supernatant also increased with increasing LAA concentration, a further result that validates the above assumptions.

### Example 5

### Investigation of the Thermal Stability of the LAA-Montmorillonite Chelate

First, 0.1000 g samples of the chelated clays were prepared as described in previous examples. Then to determine the thermal stability, 0.1000 g samples of 0.5000 g calcium montmorillonite chelated with 0.0500 g, 0.1000 g, 0.2500 g and 0.5000 g of LAA, were heated in an oven at 90° C. for 30 minutes. Pellets of the cooled samples were prepared with fused KBr and FTIR spectra of these samples were obtained. Reference spectra of the calcium montmorillonite chelated clays before heating were also obtained for comparison. Next, 0.1000 g samples of the 0.5000 g calcium montmorillonite chelated with 0.2500 g of LAA were heated at 100, 120, 140, 160, 180, 200° C. for 30 minutes. After cooling, pellets were prepared with fused KBr and FTIR spectra of the samples were obtained. Reference spectra of unchelated LAA at room temperature and when heated to 90° C., 120° C., 140° C., 160° C., 180° C., 200° C. for 30 minutes were also obtained.

The results shown in FIG. 10 and FIG. 11 indicated that these samples are thermally stable in the temperature range of about 90° C. to about 200° C.

### Example 6

### Study of Rate of Release of LAA at Different pH Values from Calcium Montmorillonite Chelate

First, a 0.2500 g sample of 0.2500 g LAA-calcium montmorillonite chelate was enclosed in a dialysis bag made of low migrant poly (vinylchloride) and placed in a 3-neck round-bottom flask. Distilled water (50.00 cm³) whose pH was adjusted to 3.0 using conc. HCl (aq), was added to the flask along with a piece of activated zinc. A balloon containing N₂ gas was attached to one of the necks and a pipette was attached to another neck. The flask was placed in a water bath and the temperature was adjusted to 25.0° C. This setup was placed on the orbital shaker tray and was shaken at 50 rpm. After 30 minutes, a 5.00 cm³ aliquot was removed and its absorbance was measured at 250.0 nm using the UV-visible spectrophotometer. Distilled water was used as the reference. The aliquot was returned to the flask, and after another 30 minutes, that is, 1 hour after initiation of the experiment, the absorbance of another aliquot was measured. This aliquot was returned and, using the same method, readings were taken at 2, 4, 6, 8, 10 hours after initiation of the experiment.

The same procedure was carried out using distilled water at pH of 5.0 and 7.0. But, absorbance values were measured at 265.6 nm and 265.0 nm, respectively. The procedure was also carried out at pH of 9.0, but aliquots were taken after 30 minutes, 2 hours, 3½ hours and 5½ hours and the absorbance was measured at 265.0 nm. Also, the procedure was carried out at pH of 1.0, but the volume of distilled water used was 41.50 cm³.

The leaching results of the LAA-calcium montmorillonite chelates at various pH values are shown in FIG. 9.

### Example 7

### Calculated Projection of Levels of LAA that is Released in the Human GI Tract

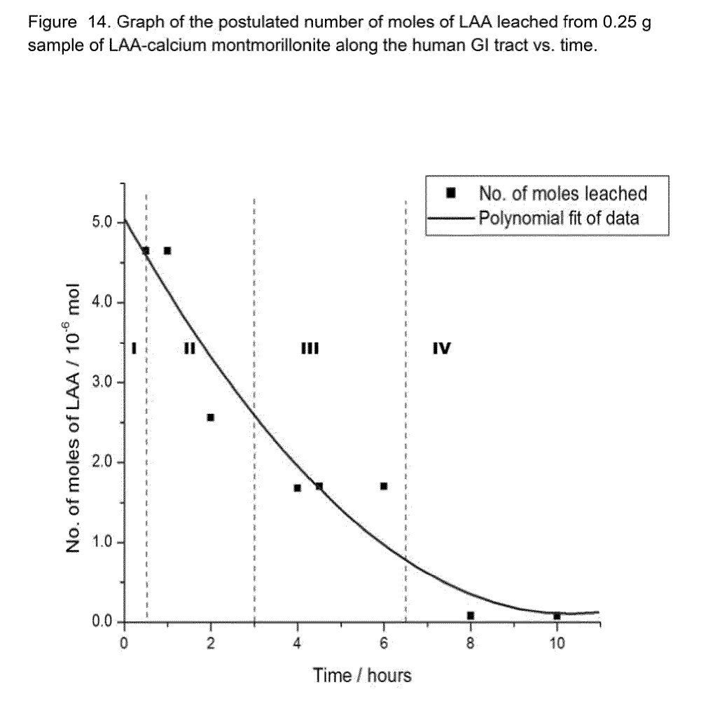

Using the data from example 7, a projection LAA release from a calcium montmorillonite chelate occurring within the human GI tract is shown in FIG. 14. In this calculated projection transit times given in Table 5 that occur within the organs of the GI tract is used. The release rates of the LAA at pH ranges found in the human GI tract indicates that the LAA-montmorillonite chelate can be used in sustained-release of LAA.

TABLE 5
Average pH values and transit times of the human GI tract

| Section of the human GI | | | |
|---|---|---|---|
| tract | Average pH | Transit times | |
| Mouth and oesophagus | 7 | 10-20 | minutes |
| Stomach | 1-3 | 2½ | hrs |
| Small intestines | 6 | 3½ | hrs |
| Large intestine | 5.5-7 | Upto 15 | hrs |

For the projection, it is assumed that the LAA-calcium montmorillonite chelate reaches the stomach within 30 minutes and before that time only a negligible amount of LAA leaches out due to the time factor and the pH in these areas being close to 7.0. Next, it is considered that the LAA-calcium montmorillonite chelate remains at a pH of 1.0 in the stomach until 2 hours after ingestion. Then, as the LAA-calcium montmorillonite chelate moves slowly out of the stomach and into the small intestine, the pH gradually changes through 3.0 to 5.0. The LAA-calcium montmorillonite chelate spends about 3½ hours in the small intestines at around this pH (region III) and then moves into the large intestine, where the pH rises to 7.0 (region IV). Greatest amount of leaching of LAA occurs in the regions where absorption takes place (stomach and small intestine) and this is beneficial. Also, since the release of LAA is sustained, there is a sufficiently high concentration of LAA present constantly in the blood. From the calculations no more than 1×10⁻⁵ mol of LAA is released in total from the 0.2500 g sample of LAA-calcium montmorillonite chelate. Accordingly, the recommended daily intake limit of 90 mg (5.1×10⁻⁴ mol) of LAA (or Vitamin C) is not exceeded.

1. A thermally stable Vitamin C composition comprising montmorillonite, an organic acid comprising L-ascorbic acid or a derivative thereof, a divalent cation present within nanolayers of the montmorillonite, wherein the organic acid is combined with the divalent cation within the nanolayers to form an organic acid-montmorillonite chelate, and wherein the said organic acid-montmorillonite chelate is thermally stable compared to L-ascorbic acid.

2. The composition of claim 1 wherein the divalent cation is selected from a member of the group consisting of calcium, magnesium, zinc, barium, and combinations thereof.

3. The composition of claim 1 wherein the organic acid is selected from a member of the group consisting of caffeic acid, quinic acid, chlorendic acid, citric acid, methionine, cysteine, malic acid, dimercapto succinic acid, aspartic acid, orotic acid, and combinations thereof.

4. The composition of claim 1 wherein the derivative of ascorbic acid is: 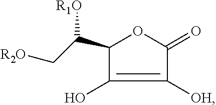
wherein R₁ and R₂ is independently selected from a member of the group consisting of linear or branched alkyl having 1 to 20 carbon atoms, an alkylcarbonylmethyl, an alkoxycarbonyl methyl, an alkyl carbonyl ethyl, an alkoxy carbonyl ethyl, an allylalkyl, an acyl, a sulphonic acid group, an phosphoric acid group, and hydrogen, with the proviso that both R₁ and R₂ are not hydrogen.

5. The composition of claim 2 wherein the L-ascorbic acid is combined with the divalent calcium cation to form a L-ascorbic acid-montmorillonite chelate.

6. The composition of claim 5 wherein the L-ascorbic is released from said L-ascorbic acid- montmorillonite chelate in the pH range of about 1 to about 5.

7. The composition of claim 5 wherein at least 95% of said L-ascorbic acid-montmorillonite chelate is formed at about a pH of about 9.

8. The composition of claim 5 wherein said L-ascorbic acid-montmorillonite chelate is thermally stable in the temperature range from about 90° C. to about 200° C.

9. The composition of claim 5 wherein said L-ascorbic acid-montmorillonite chelate is thermally stable in the temperature range from about 140° C. to about 200° C.

10. The composition of claim 5 further comprising a filler and pharmaceutically acceptable excipients.

11. The composition of claim 5 wherein said L-ascorbic acid is released in the gut of a chicken at a pH about 1 to 4 when said composition is orally administered.

12. A process for preparing a thermally stable Vitamin C composition comprising:
a. contacting an organic acid comprising L-ascorbic acid or a derivative thereof with a montmorillonite containing a divalent cation present within nanolayers of the montmorillonite;
b. combining the organic acid with the divalent cation to form an organic acid-montmorillonite chelate; and
c. stabilizing the organic acid-montmorillonite chelate in the temperature range from about 90° C. to about 200° C.

13. The process of claim 10 wherein the organic acid is L-ascorbic acid and the divalent cation is calcium.

14. The process of claim 11 further comprising pelletizing the composition.

15. A method of delivering therapeutic amounts of Vitamin C to a human comprising orally administering the composition of claim 5.

16. The method of claim 14 wherein said organic acid-montmorillonite chelate is L-ascorbic acid-montmorillonite chelate and the divalent cation is calcium.

17. The method of claim 16 further comprising sustained releasing of Vitamin C from said L-ascorbic acid-montmorillonite chelate in the stomach of a human in the pH range from about 1 to about 5.

18. The method of claim 17, further comprising sustained releasing of calcium cations from said L-ascorbic acid-montmorillonite chelate in the stomach of a human in the pH range from about 1 to about 5.
